(19) United States
(12) Patent Application Publication
(10) Pub. No.: US 2015/0216414 A1
(43) Pub. Date: Aug. 6, 2015

(54) Measuring Information Acquisition Using Free Recall

(71) Applicant: The Schepens Eye Research Institute, Inc., Boston, MA (US)

(72) Inventors: Russell L. Wood, Quincy, MA (US); Daniel R. Saunders, Cambridge, MA (US); Peter Bex, Concord, MA (US)

(21) Appl. No.: 14/426,314

(22) PCT Filed: Sep. 11, 2013
(86) PCT No.: PCT/US13/59109

§ 371 Date: Mar. 5, 2015

Related U.S. Application Data
(60) Provisional application No. 61/700,111, filed on Sep. 12, 2012.

(51) Int. Cl. A61B 5/00; A61B 3/02; A61B 5/12; A61B 5/16

(52) U.S. Cl. CPC A61B 5/0048; A61B 5/168; A61B 3/02; A61B 5/123; A61B 5/4011; A61B 5/4803

(57) ABSTRACT

Assessing acquisition of information related to a stimulus can be accomplished by providing a stimulus, recording a free recall response in natural language of the stimulus by a subject, and determining automatically a similarity. The similarity is between the recorded free recall response and a database comprising one or more control responses associated with the stimulus. A higher similarity indicates greater information acquisition by the subject. Related apparatus, systems, techniques, and articles are also described.

### RELATED APPLICATIONS

The present application claims priority to US Provisional Patent Application No. 61/700,111, filed Sep. 12, 2012, the contents of which are hereby incorporated by reference in its entirety.

### TECHNICAL FIELD

The subject matter described herein relates to measuring information acquisition by subjects using free recall responses to stimuli.

### BACKGROUND

Free recall is a strategy typically used in the study of memory. Free recall asks participants to inspect or be subject to stimuli, and then the participants are prompted to describe (e.g., recall) the stimuli using their own words. The recall can be written or spoken. For example, a participant could study a list of items, and then the participant is prompted to recall the list in any order. Often the recall period starts immediately after the final list item; this can be referred to as Immediate Free Recall (IFR) to distinguish it from Delayed Free Recall (DFR). A short distraction period can be included and the free recall response can be a short verbal response. Free recall can also involve reporting as many details as can be recalled of the stimulus or stimuli. It can also involve reporting by the participant of the gist or meaning of a stimulus or stimuli or describing the stimulus or stimuli, which is also known as an open response. For example, a participant may read a passage of text and then be asked to describe the passage in their own words.

Cognitive and sensory (e.g., vision, hearing, and the like) impairment are issues of increasing concern. The prevalence of these impairments increase with age, and the current population among developed nations is aging. People with mild cognitive impairment develop dementia at a much higher rate than healthy people of the same age. Recent military conflicts have left a larger proportion of survivors with cognitive impairments than in the past. Most people with vision impairment report difficulty reading, recognizing faces, and watching television and movies, and extreme difficulty with video on handheld devices. Hearing impairment can cause difficulties in many settings including social interactions, driving and watching television and movies.

### SUMMARY

In one aspect, assessing acquisition of information related to a stimulus can be accomplished by providing a stimulus, recording a free recall response of the stimulus by a subject, and determining automatically a similarity. The similarity may be between the recorded free recall response and a database comprising one or more control responses associated with the stimulus. A higher similarity may indicate greater information acquisition by the subject.

In another aspect, data may be received characterizing a free recall response of a stimulus by a subject. A similarity may be automatically determined between the recorded free recall response and a database comprising one or more control responses associated with the stimulus. A higher similarity may indicate greater information acquisition by the subject. The similarity may be provided.

In yet another aspect, data may be received characterizing a free recall response of a stimulus by a subject and a database comprising one or more control responses associated with the stimulus. A similarity may be automatically determined between the recording free recall response and the database. A higher similarity may indicate greater information acquisition by the subject. The similarity may be provided.

In some variations, one or more of the features disclosed herein including the following features can optionally be included in any feasible combination. The stimulus can be one or more of visual, auditory, olfactory, and tactile. The assessment of acquisition of information can be used for one of the following: assessment of a subject's high-level vision; assessment of conditions that impair vision; assessment of conditions that impair hearing; assessment of impairments olfaction; assessment of conditions that impair tactile sensory function; assessment of conditions that impair cognitive function; assessment of treatments of vision disorders; assessment of treatments of hearing disorders; assessment of treatments of olfaction disorders; assessment of treatments of cognitive disorders; assessment of a quality of the stimulus; assessment of the subject's affinity for the stimulus; and assessment of an effectiveness of image, video, or audio compression algorithms.

The assessment can be used to evaluate the subject's interest in the stimulus. The similarity can be determined using natural language processing (e.g., cognitive linguistics). The similarity can be determined by counting a number of words in the recorded free recall response that are contained in the database of control responses, with a higher count indicating a greater similarity and a greater acquisition of information.

The similarity can be used to evaluate one or more characteristics of the subject. The evaluation can be one or more of the following: an assessment of visual function; an assessment of a disorder affecting visual function; an assessment of auditory function; an assessment of a disorder affecting auditory function; an assessment of olfactory function; an assessment of a disorder affecting olfactory function; an assessment of tactile function; an assessment of a disorder affecting tactile function; an assessment of cognitive function; an assessment of a disorder affecting cognitive function; an assessment of the outcome of a medical intervention; and an assessment of the subject's attention.

The disorder affecting visual function can be selected from a group consisting of: tears, cornea, conjunctiva, crystalline lens, retinal degeneration, subretinal degeneration, dry eye, cataract, glaucoma, amblyopia, macular degeneration, retinitis pigmentosa, diabetic retinopthy, optic neuritis, acquired brain injury, and traumatic brain injury.

The disorder affecting hearing function can be selected from a group consisting of tinnitus, sensorineural hearing loss, vestibulocochlear nerve damage, conductive hearing loss, sensorineural hearing loss, central hearing loss, functional hearing loss, and mixed hearing loss.

The disorder affecting olfaction can be selected from a group consisting of: anosmia, dysosmia, hyperosmia, hyposmia, olfactory reference syndrome, parosmia and phantosmia.

The disorder affecting tactile function can be selected from a group consisting of tactile sensory deficits, allodynia, hyperalgesia and nerve injury.

The disorder affecting cognitive function can be selected from a group consisting of: autism, dyslexia, dyscalculia, attention deficit disorder (ADD), schizophrenia, multiple sclerosis, stroke, mild cognitive impairment, dementias, Alzheimer's disease, acquired brain injury, and traumatic brain injury.

The similarity can be used to evaluate one or more characteristics of the stimulus. The evaluation can be one or more of the following: an assessment of image quality; an assessment of video quality; an assessment of audio quality; an assessment of a compression and/or decompression algorithm; an assessment of one or more compression and/or decompression algorithm settings; an assessment of a stimulus presentation device quality; an assessment of the effectiveness of image; an assessment of a video enhancement algorithm; an assessment of an audio enhancement algorithm; an assessment of an enhancement algorithm settings; and an assessment of the ability of a symbol to transmit its intended message.

The similarity can be used to evaluate at least one device, method, or system that modifies the stimulus prior to the provision of the stimulus. The similarity can be used to evaluate at least one device, method, or system that modifies the stimulus after the provision of the stimulus and prior to the recording of the free recall. The device can be an assistive device.

The stimulus can be selected from a group consisting of: video, audio recording, image, smells, tactile stimulation, such as sensory substitution devices including BrainPort, and text that is written, spoken, presented as Braille, Rapid Serial Visual Presentation or in codes such as morse or semaphore. The recorded free recall response can be an audio recording of speech that is manually translated to text or automatically translated to text using a speech recognition program.

Providing the similarity can include at least one of displaying, storing, persisting, processing, and transmitting.

Computer program products are also described that comprise non-transitory computer readable media storing instructions, which when executed by at least one data processors of one or more computing systems, causes at least one data processor to perform operations herein. Similarly, computer systems are also described that may include one or more data processors and a memory coupled to the one or more data processors. The memory may temporarily or permanently store instructions that cause at least one processor to perform one or more of the operations described herein. In addition, methods can be implemented by one or more data processors either within a single computing system or distributed among two or more computing systems.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a system for assessing acquisition of information related to a stimulus;

FIG. 2 is a process flow diagram illustrating a method of assessing acquisition of information related to a stimulus;

FIG. 3 is a plot illustrating a drop in shared word score due to image-processing blur condition;

FIG. 4 is a plot illustrating a drop in shared word score due to visual acuity condition;

FIG. 5 is a table comparing the demographics of the two control samples, and the control samples to the demographics of the United States as a whole;

FIG. 6 are plots A and B illustrating the distribution of response lengths, after removing frequently-occurring words, between the in-lab and crowdsource responses;

FIG. 7 are plots A, B. and C illustrating the mean number of words shared by responses with responses to the same clip (filled bars), and with responses to other clips (open bars); and

FIG. 8 is a plot illustrating the difference in mean shared word score between people with normal vision, people with central vision loss, and people with hemianopia.

### DETAILED DESCRIPTION

Information acquisition can be measured by providing a subject with a stimulus, allowing the subject to perceive the stimulus, and recording a free recall response to the stimulus by the subject. For example, the subject can view a video or audio clip and describe (e.g., by speaking or writing) the stimulus in their own words. The recorded free recall response can be compared to a database of control responses, and a measure of similarity between the recorded free recall response and the control database can be determined. The closer or more similar the recorded response is to the control database, the greater the information acquisition of the subject. The measure of information acquisition can then be used to assess, for example, characteristics of the subject or stimulus.

In general, the quality of perception by a subject of a stimulus can directly relate to the amount of information that transfers to the subject. If the quality of perception of the stimulus is high, the subject may obtain more information. Conversely, if the quality of perception of the stimulus is low, the subject may obtain less information. The quality of the stimulus can affect the perception of the stimulus.

In other words, if the ability of a subject to perceive a stimulus is inhibited (for example, by poor eyesight, hearing, cognitive function, and the like) the subject will learn less about the stimulus. If, on the other hand, the stimulus is of poor quality, the subject will also learn less about the stimulus.

The subject's cognitive, auditory, olfactory, tactile, or visual system or the stimulus quality can affect information. By measuring information acquisition, the subject's cognitive, auditory, olfactory, tactile, and/or visual system, or the stimulus quality can be assessed or evaluated. For example, the current subject matter can evaluate a subject's high-level visual function because visual problems can be reflected in a discrepancy of a response from the normally sighted control responses. This discrepancy can reflect missing information and/or inaccurate information. In either case, it is a failure of information acquisition.

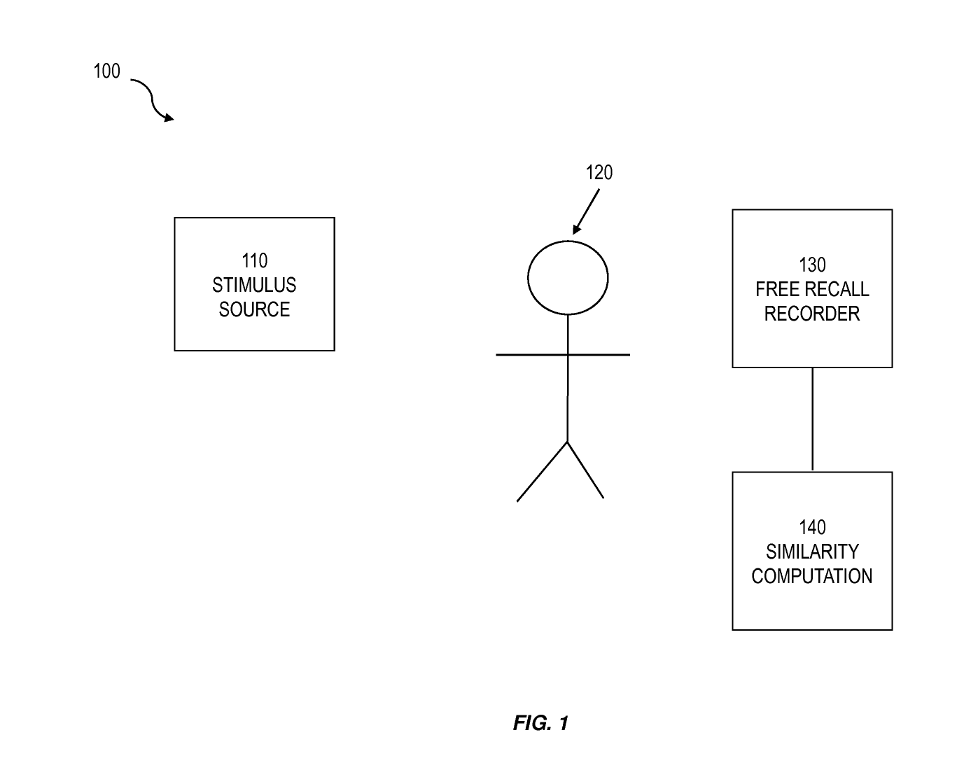

FIG. 1 is an example implementation of a system 100 for assessing acquisition of information related to a stimulus. A stimulus source 110 provides the stimulus. In one example implementation, the stimulus can be visual, audio, or a combination of visual and audio, such as video. For example, a television or personal computing device can be a stimulus source. The stimulus source 110 can provide the stimulus to a subject 120. The subject 120 can perceive the stimulus (by viewing, listening, watching, and the like) and can provide a free recall of the stimulus. The free recall can be a description of the stimulus by the subject 120 using his or her own words. A free recall recorder 130 can record the free recall. The recording can be, for example, an audio recording of the free recall communicated by the subject 120 (e.g., an audio clip of speech) and/or can be, for example, text written or typed by the subject 120. The free recall recorder 130 can be an audio recording device with microphone and storage capabilities and can include a personal computing device such as a smartphone, tablet, personal computer, and/or other device. If speech is recorded, a speech to text program can be utilized to automatically translate the free recall speech into a written (e.g., text) representation of the free recall or the audio recording can be transcribed by a human operator.

Similarity computation processor 140 can determine a similarity between the recorded free recall response and one or more control free recall responses associated with the stimulus. These control responses may be stored in a database.

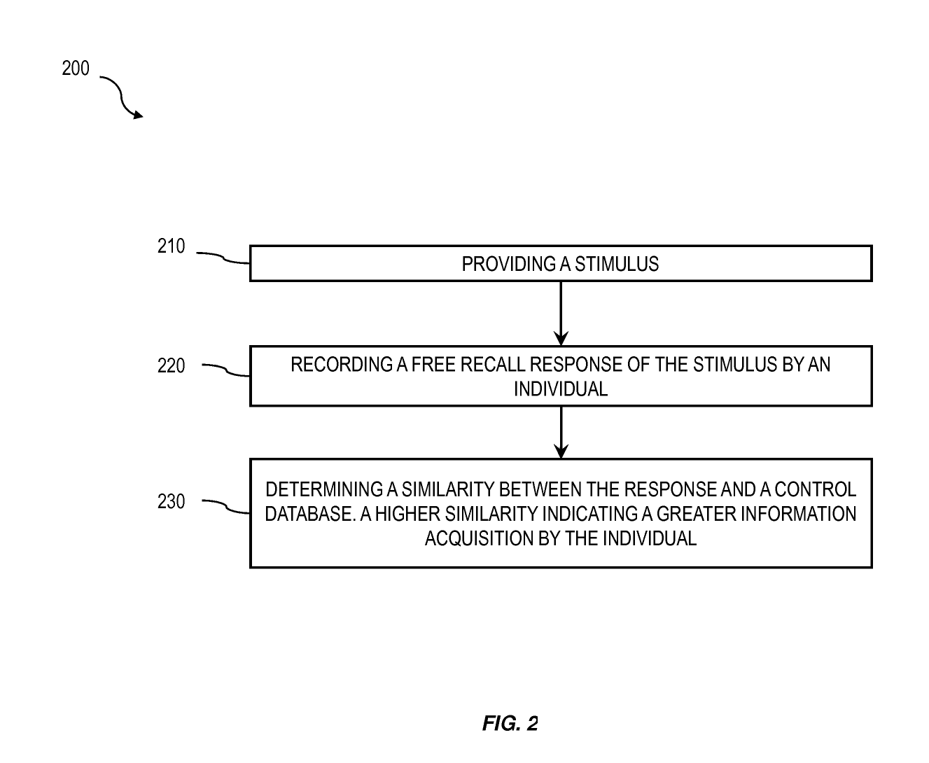

FIG. 2 is a process flow diagram 200 illustrating a method of assessing acquisition of information related to a stimulus. At 210, a stimulus can be provided. The stimulus can be provided to a subject 120. The subject 120 can perceive the stimulus and provide a free recall response. For example, a stimulus may include one or more static images of a scene (e.g., a picture of a ball) and prompt a free recall response describing characteristics of the scene (e.g., color of ball, apparent texture, relative size, and the like).

At 220, the free recall response provided by the subject 120 can be recorded. For example, the recording can be audio (e.g., of speech) or text, although other responses may be implemented as well. If the recording is audio, the audio can be converted to text using speech recognition software or other converter.

At 230, a similarity between the free recall response and a control database can be determined. The control database can include one or more control free recall responses or data derived from one or more control free recall responses. The one or more control free recall responses can be taken from other subjects who have previously viewed the same or similar stimulus. The control free recall responses can be collected under “normal” or “control” conditions. The database of control free recall responses (also referred to as a control database) can be considered a reference baseline to which the recorded free recall response of 220 is compared. A greater similarity between the recorded free recall response and the database can indicate greater information acquisition by the subject 120 and similarity to the control group.

The “control” conditions can depend on the characteristics that the assessment of acquisition of information is intended to evaluate. For example, to evaluate a high-level visual function of a subject, the “control” database can include control free recall responses from a plurality of subjects provided with the same stimulus and evaluated to have normal vision (e.g., “20/20” vision) and normal cognitive function as measured by another assessment tool such as the Montreal Cognitive Assessment, Weschler Intelligence Scale, or the Woodcock Johnson Tests of Cognitive Abilities. Another example of a control group could be military personnel who have not experienced an event that could lead to traumatic brain injury. Another example of a control group could be people with normal olfaction and cognition. Thus, the similarity of the recorded free recall response to the control database can be a measure of high-level visual function as compared to the plurality of “control” subjects.

Similarity can be determined using natural language processing, computational linguistics, and/or other numerical or statistical approach. Natural language processing relates to techniques for enabling computers to derive meaning from human or natural language input. For example, an algorithm for determining the similarity can comprise counting the number of words in the recorded free recall response that are contained in each of the control responses within the database of control responses and averaging the count. A higher average count would indicate a greater similarity.

Latent Semantic Analysis (LSA) is another technique of natural language processing. LSA is a technique utilizing vectorial semantics, and includes analyzing relationships between a set of documents and the terms they contain by producing a set of concepts related to the documents and terms. LSA assumes that words that are close in meaning will occur in similar pieces of text. A matrix containing word counts per paragraph can be constructed from the recorded free recall and each of the control database responses, although other mathematical techniques can be used to compare the matrices to determine a similarity (or distance).

Perception can affect information acquisition. Perception is a complex process by which information received from sensory organs is organized, identified, and interpreted in order to fabricate a mental representation of physical stimulation. All perception involves signals in the nervous system, which in turn result from physical stimulation of the sense organs. For example, vision involves light striking the retinas of the eyes. Perception is not the passive receipt of these signals, but is shaped by cognitive functions such as learning, memory, and knowledge. Perception includes low level factors to build up higher-level information (e.g., to recognize an image of a basketball, one must first perceive a sphere) as well as high level factors that include a person's knowledge and expectations that influence perception (e.g., to recognize an image of a basketball, one must first know what a basketball is). Perception depends on complex functions of the nervous system, but subjectively seems mostly effortless because this processing happens outside conscious awareness.

In the case of visual stimulus, acquisition of information from a scene is a function of the viewer's perception, which can be affected by, among other factors, the viewer's cognitive system, the viewer's visual system, and the quality of the scene. Broadly, a scene is a visual stimulus. A scene may be a natural or constructed (e.g. drawn, painted, computer generated), it may be a view of the real world, or it may be (static) images or video (sequential images). High-level visual function incorporates many aspects of vision and influences many activities of daily living. Understanding a scene is an everyday, almost constant, use of vision. Similarly, for auditory stimuli, understanding of auditory scenes (events) is an activity of daily living.

Similarly, acquisition of information from written text is a function of the reader's perception, which can be affected by, among other factors, the reader's cognitive system, the reader's visual system, and the quality of the text. Likewise, acquisition of information from spoken words depends on the listener's perception and can be affected by, among other factors, the listener's cognitive function, auditory function and the quality of the spoken words. Acquisition of information from text presented as Braille or Rapid Serial Visual Presentation also calls on various aspects of sensory and cognitive systems and display quality.

A scene may also be natural sounds, music, constructed sound patterns, smells, or a tactile stimulation pattern, such as Braille or sensory substitution input. Understanding the meaning of such stimuli are activities of daily living.

The current subject matter can be applied to evaluate a number of characteristics. The characteristics can be of the subject, such as elements of perception (auditory function, visual function, cognitive function, and the like). The current subject matter can be used as a diagnostic test for diseases or disorders affecting perception, to monitor progress of a condition, and/or as an outcome measure for medical interventions (e.g., to assess therapeutics). For example, macular degeneration, retinitis pigmentosa, and traumatic brain injury can affect high-level vision and be assessed using the current subject matter. Additionally, Alzheimer's disease, and traumatic brain injury can affect cognitive function and can be assessed using the current subject matter.

The characteristics of the subject can include the affinity, interest, attention, and/or engagement of the subject for the stimulus. For example, a subject can watch an episode or short clip of a television, radio, or other media program and provide a free recall response. The information acquisition by the subject can be assessed using the current subject matter. A greater information acquisition by the subject would indicate a greater affinity, interest, attention, and/or engagement of the subject to the media program. This could be used as an indication of future success of the media program. Thus, the current subject matter can be used as a supplement or substitute to Nielson ratings.

The characteristics can be of the stimulus, such as a quality of an image, video, text, audio, olfactory, tactile, or video stimulus. For example, the current subject matter can assess the video or audio compression and/or decompression algorithms and settings of the algorithms. The greater the acquisition of information by the subject (as compared to, for example, a control database of subjects who have viewed an uncompressed or high-resolution version of the stimulus) the better the assessment of the video or audio compression algorithm. The characteristics of the stimulus can include the stimulus presentation device. For example, the current subject matter can be used to evaluate a projector, head-mounted display, visual or auditory prosthesis and the like. Additionally, the assessment can be of the ability of a symbol (e.g. road sign, icon, Braille character) to transmit its intended message.

The characteristics can be of a device, method, or system that modifies the stimulus after the provision of the stimulus but prior to perception by the subject, such as an assistive device. For example, an assistive device can include corrective glasses, a hearing aid, or a device that provides enhancement of video for low vision rehabilitation patients. Thus, the assistive device modifies the stimulus prior to the recording of the free recall response. The assistive device can include sensory substitution or prosthetic devices.

The stimulus can be presented and the responses can be recorded in an automated manner (e.g. computer, handheld device, voice recognition).

As used herein, a stimulus can include one or more stimulus (e.g., stimuli). For example, a video can include both visual and audio stimulus but, in general, the video will be referred to herein as a stimulus, even though it can contain multiple components such as an audio component and a video component. In general, the stimulus can be anything that stimulates human perception (smell, touch, taste, body balance, acceleration, temperature, pain, time, and the like).

Without in any way limiting the scope, interpretation, or application of the claims appearing below, a technical effect of one or more of the example implementations disclosed herein may include a test of cognition that involves low and high level factors thus being sensitive not only to difficulties in acquiring details from the stimulus (e.g. visual viewing, listening), but also to difficulties in processing dynamic information and understanding of the factual content of the stimulus. Moreover, without in any way limiting the scope, interpretation, or application of the claims appearing below, another technical effect of one or more of the example implementations disclosed herein may be not requiring the formulation of a quiz to assess high-level visual, audio, and cognitive functioning and thus is not subject to the bias of the people who construct the questions. Additionally, without in any way limiting the scope, interpretation, or application of the claims appearing below, another technical effect of one or more of the example implementations disclosed herein may be that the current subject matter is more sensitive because content quizzes are labor intensive to create and are subjective.

Without in any way limiting the scope, interpretation, or application of the claims appearing below, another technical effect of one or more of the example implementations disclosed herein may be an easy-to-administer measure of high-level visual, auditory, and cognitive function as well as provide a new outcome measure that is more representative of the vision, auditory, olfactory, tactile and cognitive function required for activities of daily living than current measures. Moreover, without in any way limiting the scope, interpretation, or application of the claims appearing below, another technical effect of one or more of the example implementations disclosed herein may be novel measures for treatments of conditions that impair one or more of visual, auditory, olfactory, tactile sensory and cognitive function (such as Alzheimer's disease). Additionally, without in any way limiting the scope, interpretation, or application of the claims appearing below, another technical effect of one or more of the example implementations disclosed herein may be providing a method of determining when an audio or video compression and/or decompression algorithm produces an experience that is acceptable to a viewer or listener, as well as evaluating the effects of different procedures or algorithms on the experience. For example, an enhancement to improve the quality of video compressed due to bandwidth limitations can be evaluated.

The following provides an example related to measuring information acquisition using free recall.

It is shown that reducing the scene quality by introducing blur through image processing produces a greater reduction in information acquisition (worse performance) as the blur increases. Similarly, it is shown that reducing the visual quality by defocus (blurring) produces a greater reduction in information acquisition as the defocus increases.

A large set of video clip descriptions from normally-sighted subjects was collected. There were 60 participants, with equal numbers of three age groups: under 60 years old, 60-70y, and greater than 70y, each with equal numbers of men and women. They all had binocular visual acuity better than or equal to 20/30 and no ocular conditions in self-reported ophthalmologic history. 200 video clips of 30 s duration were selected from Hollywood films and nature TV programs, with several genres represented. Participants saw 40 clips each, leading to a total of 12 responses per video clip, and 2400 responses in total. They saw two prompts, “Describe this movie clip in a few sentences, as if to someone who has not seen it” and “Is there any other detail you want to mention?”, and the audio of the two responses were automatically concatenated to make the final response. The responses were automatically transcribed using the speech recognition program MacSpeech Scribe v1.1 (2010). These transcriptions were then corrected by a separate set of Amazon Mechanical Turk workers.

In the scoring algorithm, a response was scored by comparing it to control responses to the same video clip (in this example, 12 control responses). The more similar the new response, the better the score, so that a response that had no overlap with what normally-sighted people mentioned about the clip received the lowest possible score, whereas a response that includes many frequently-mentioned features of the clip received a high score. Several algorithms were evaluated for computing text passage similarity. The evaluation was based on a take-one-out procedure: for each response in the control database, the response was removed it from the database, and scored based on the remaining database as if it were a new response. If the score could be used to correctly classify the response according to the originating video clip, that is, of all the 200 clips it had the highest average similarity with the one it was associated with, then that was counted in the algorithm's favor. Therefore, text-based similarity algorithms were compared based on their percent correctly classified.

Several text passage similarity metrics were derived from computational linguistics. The text was processed with the Text to Matrix Generator toolbox for MATLAB. In all cases, a list of stop words was first removed from the text passage, consisting of less informative words such as “of” and “the,” as well as verbal interjections such as “um” and “sorry.” The first approach to passage similarity evaluated was Latent Semantic Analysis, which is based on singular value decomposition of the frequency matrix of words occurring in text passages. When two words co-occur in a passage, the algorithm brings them closer in semantic space, as well as the words that co-occur with each of the words in other passages. As described by Landauer and Dumais (Landauer T K, Dumais S T. A Solution to Plato's Problem: The Latent Semantic Analysis Theory of Acquisition, Induction, and Representation of Knowledge. Psychological Review. 1997; 104(2):211-240), “LSA represents the meaning of a word as a kind of average of the meaning of all the passages in which it appears and the meaning of a passage as a kind of average of the meaning of all the words it contains.” LSA has been previously used successfully to grade student essays by comparison to a master essay, and to compare scientific abstracts.

From example 1, the highest rate of correct classification, that is, matching responses to the video clip of origin, was the simple count of average shared words. Unlike the other algorithms it does not have a mechanism for dealing with synonyms, such as “river” and “stream.” Since the words do not match, they will not increase the number of shared words. Nor does the algorithm explicitly deal with word endings, counting “read” and “reading” as two unrelated words. However, with a large enough baseline, several synonyms for a concept will naturally occur among the responses, which increases the chances that the wording of the concept will be recognized in a new response. Furthermore, while LSA and other algorithms deal with synonyms, they may have found accidental synonyms, contributing noise to the scores. Whatever the reason, the shared word score was the best of the set of published algorithms tested, and achieved high classification performance.

In another algorithm, the way that a response is evaluated is to count the number of non-repeating words that appear in both the target response and each of the 12 control responses (11 from the video clip that the response originated from), after removal of stop words. Then the score is the average of these shared word counts. Therefore, if the same word appears in multiple control responses, it will be effectively weighted more highly, whereas multiple occurrences of the same word within a control response will not increase the score.

To evaluate if a smaller control database would still be effective the percent correctly classified control databases with fewer than 12 responses per video clip was computed, by randomly sampling n responses for each video and recomputing the percent of responses that were correctly classified, again using a take-one-out strategy. The randomization procedure was repeated 100 times per value of n, ranging between 2 and 11. The percent correctly classified increased until it began to plateau at around 8 responses and 90% correct. As another way to evaluate smaller control databases, the error in the score of a particular response with a particular randomly sampled was estimated as the difference between the computed score and the score with the full control database. Depending on the application, less than 12 responses per video clip in the control database can be feasible.

Two experiments were conducted to validate the average shared word score as a measure of information acquisition. They represented two distinct simulations of low visual acuity, that might be caused by cataracts, myopia, or the absence of central vision in age-related macular degeneration. In the first experiment, lowered acuity was simulated for normally-sighted participants by introducing blur through image processing.

Ninety-two workers from the Amazon.com Mechanical Turk participated in the study, with median age 31y (18 to 64y).

Twenty clips were selected from the set of 200 that were used for the control database, with each genre represented proportionally. They were processed with three levels of Gaussian blur, using a kernel with a standard deviation of 0% (no blurring), 0.8%, 1.2%, 2.4%, or 6% (where percent was related to the image width). Participants responded to the clips by typing answers to the same two free recall prompts that were used in collecting the control database.

Responses were scored by counting the average number of words in common with the 12 responses for the originating video in the control database. A mixed-model analysis was used to test for the effect of the fixed factor, blur condition, since both participant and video clip were random factors and they were fully crossed.

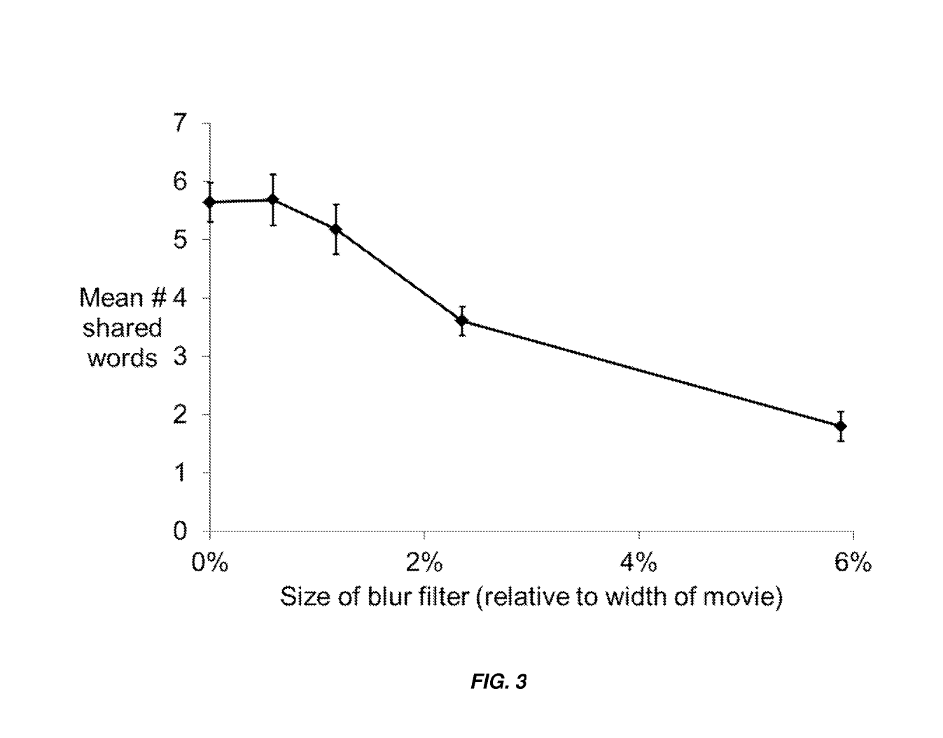

FIG. 3 is a plot illustrating a drop in shared word score due to image blur, with a significant overall difference among blur conditions, p<0.001. Posthoc pairwise tests produced by the analysis, and adjusted for multiple comparisons using the Sidak correction, showed that all levels were well differentiated from one another, p<0.005, with the exception of 0 and 0.8 (p=0.989) and 0.8 and 1.2 (p=0.140).

In the second experiment, lowered visual acuity was created by having participants wear different strengths of defocus lenses while they viewed a subset of video clips and gave responses as in the control data collection. Lower levels of visual acuity, induced by the defocus lenses, produced lower shared word scores.

Fifteen participants from the community were recruited, with median age 34y (21 to 67y), and reporting normal or corrected-to-normal vision. Spherical defocus lenses were selected for each participant that produced visual acuities of 20/20, 20/50, 20/125, 20/320, and 20/800.

The same twenty clips selected from the set of 200 were used for the second experiment. Each participant viewed all the clips in random order, looking through defocus lenses that were switched between each trial, for a total of 4 trials for each acuity condition. They were given the same two prompts asking for a description of the movie as in the control data collection, and their verbal responses were also transcribed using MacSpeech Scribe and Mechanical Turk workers as for the control participants as described above. Responses were scored and analyzed as in the image processing blur experiment.

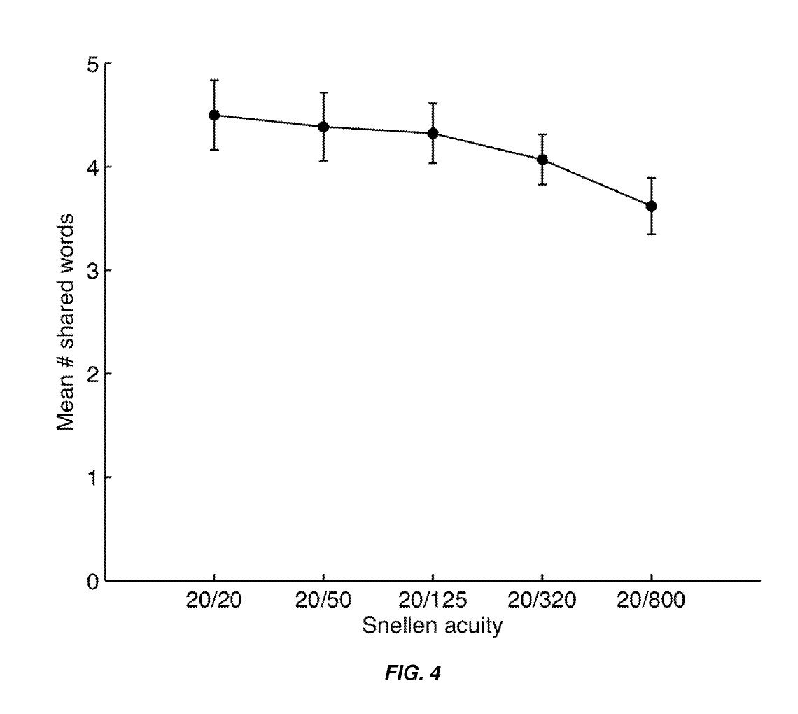

FIG. 4 is a plot 400 illustrating a drop in shared word score due to acuity condition, with a significant overall difference among the acuity levels, p<0.001. The mean number of words shared by responses with responses to the same clip for different levels of acuity is shown, where a higher number indicates worse visual acuity and therefore more degraded vision. Error bars indicate 95% confidence intervals. Comparing all conditions to the 20/20 acuity condition, the 20/800 condition scoring significantly lower, p<0.001, and so did the 20/400 condition, p=0.038. The shared word scores in the other acuity conditions were not significantly different from the 20/20 condition.

The shared word measure was capable of detecting an effect of lowered acuity with 60 responses per acuity condition. The decreased acuity certainly lowered the amount of information in the video clip that was available to the viewer, and so this provides support for the idea that the average number of shared words is a valid measure of information acquisition from video clips.

The example 1 described herein is an approach to evaluating perception of video that does not rely on subjective impressions or experimenter-created scoring keys, and applies to watching TV and movies for recreation. While the process of visual information acquisition from video is an extremely complicated and multi-stage process, it is known that when there is less information in the image, as in the image processing and the defocus experiment, less information will be acquired. Therefore, the results of the image processing and the defocus experiment show that it is effective at measuring information acquisition.

The following describes another example related to measuring information acquisition. The second example describes two large databases collected using different methods for use as control databases. One of the databases was collected using crowdsourcing, which is shown to be an effective way to collect a control database.

Internet-based crowdsourcing of medical studies has had a number of successes in recent years. 20,000 members of the 23 and Me genome-sequencing community responded to a detailed survey about their phenotype. Over 500 subjects with developmental prosopagnosia were identified through self-testing on the Web site faceblind.org, and thousands of online participants contributed information about their off-label drug use. These studies would have been prohibitively expensive and time-consuming to conduct through traditional recruiting and testing. For these examples, the majority of the data consist of categorical responses. However, for many purposes it would be valuable to collect large natural language databases, related to a specific prompt, over the Internet using crowdsourcing. For example, to norm projective psychological tests, or to compile qualitative descriptions of disease symptoms. As discussed above, a measure of information acquisition can be used to quantify the benefit of video enhancements for people with low vision. Rather than scoring the content of the responses manually, an algorithm can be used that automatically compares newly received text passages to a large body of control responses. In example 2, crowdsourcing is examined to determine if it is an effective way to collect this database. Specifically, do the responses provided have substantial content, and are the responses, as well as the participants giving the responses, similar to those in a supervised lab setting?

Crowdsourcing, refers to the practice of making work available to an unspecified pool of workers, usually by posting an open call on the internet. Workers are typically compensated on the basis of the work they complete, rather than by a contract for a fixed amount of work. For the employer, the absence of the traditional relationship with employees, in many cases not knowing their identities or qualifications, is balanced by the speed and cheapness with which a large number of tasks can be completed. Often little time investment is required for data collection beyond the initial setup. The volume of data can compensate for potential inconsistency in quality: several studies have shown that combining the responses of non-expert workers, whether by averaging or by using majority answers to screen out low-quality answers, can equal the quality of expert work, at a much lower cost. The crowdsourcing website Mechanical Turk created by Amazon.com was utilized, because of its advertised worker base of over 500,000 subjects from 190 countries, and because of the convenient infrastructure it provides for posting and paying for small jobs (1 minute to 1 hour) to be completed over the Web. (Paolacci G, Chandler J, Ipeirotis P G. Running experiments on Amazon Mechanical Turk. Judgement and Decision Making. 2010; 5(5):411-419; and Behrend T S, Sharek D J, Measde A W, Weiber E N. The viability of crowdsourcing for survey research. Behavior Research Methods. 2011:1-14.)

Mechanical Turk and other crowdsourcing tools are particularly well suited for the task of collecting nonspecific control databases. Besides the speed and low cost of data collection, the population is relatively heterogenous, typically spanning a range of ages, educational backgrounds, and geographic locations that is greater than can be easily accessed by conventional methods. The major limitation of crowdsourcing, that it is difficult to target only people with particular demographic characteristics, is less serious when a general control database is required. However, there is still concern about whether databases collected in this way will be qualitatively different, particularly when more complex responses are requested.

A control natural language database that was collected over the Web from Mechanical Turk workers was compared with a database collected in the lab with participants recruited by conventional methods. As discussed in example 1, the responses consisted of short descriptions of 30 second movie clips. The outcomes of the two recruiting processes are compared, as well as the nature of the responses that were produced. In addition to simple metrics such as the lengths of the responses, a take-one-out procedure was used to evaluate the content. The text of each response was compared to the text of all other responses in the same control database, taking note of whether it was more similar to the responses to the same movie clip than to the responses to other movie clips, using a simple count of shared words. This procedure was also performed crossing the two control databases, to test whether the content was similar.

Crowdsourced participants were recruited through postings on Amazon.com's Mechanical Turk, and were limited to workers who were registered as living in the U.S. Demographic information was requested from each worker before they completed any tasks. At the end of the demographic survey, workers actively consented to the study by selecting a check box. Workers were only identified by an ID assigned by Amazon.com. They were paid, with Amazon.com as an intermediary, on the basis of the number of responses they provided.

In-lab participants were recruited from the community in and near Boston, Mass. using a contact list, or by being referred by participants in this and other studies. There was a target number of 60 participants divided equally into three age groups: under 60 years old, 60-70y, and greater than 70y, each with equal numbers of men and women. The age stratification ensured responses from older participants, to investigate a possible age effect and because the visual disorders our research addresses are more prevalent with age. Other criteria included normal appearance of retina, no ocular conditions in self-reported ophthalmologic history, and binocular acuity greater or equal to 20/30. Subjects were shown the clips wearing habitual, not optimal, optical correction. Participants were also rejected if their score on the Montreal Cognitive Assessment was below 20. They were compensated with a fixed payment per session, since each participant contributed the same number of responses.

There were 200 video clips selected from 39 different films and TV programs, chosen to represent a range of genres and types of depicted activities. The genres included nature documentaries (e.g. BBC's Deep Blue), cartoons (e.g. Shrek Forever After) and dramas (e.g. The Hurt Locker). The clips were 30 seconds long and were selected from parts of the films that had relatively few cuts, which was reflected in the average number of cuts per minute in our clips being 9, as compared to approximately 12 per minute in contemporary films. The clips included conversation, indoor and outdoor scenes, action sequences, and wordless scenes where the relevant content was primarily the facial expressions and body language of one or more actors. Most clips contained both factual content and emotional content. Although all participants heard audio in addition to viewing video, they were instructed to report only on the visual aspects of the clip.

Crowdsourced participants viewed the video clips within a Web browser, on a local computer of their choice. Therefore the size of the monitor, their distance from the monitor, and other display characteristics could not be determined. The clips were shown within the frame of the Mechanical Turk interface, with each clip representing a separate HIT (Human Interface Task, the unit of paid work on the Mechanical Turk website). Below the clip there were two text boxes in which to answer the two movie description prompts, “Describe this movie clip in a few sentences as if to someone who hasn't seen it” and “List several additional visual details that you might not mention in describing the clip to someone who hasn't seen it.” Crowdsourced participants could complete as many video clip description tasks as they wanted while more clips were available, at any time of day. It was not possible to guarantee that each worker would complete a certain number of these tasks. However, workers were prevented from seeing any clip more than once. Across all Mechanical Turk participants, 20 responses were collected for each clip, for a total of 4000 responses.

In-lab participants viewed the video clips on a 27-inch iMac i7 at a fixed distance of 100 cm. The videos were 33 degrees of visual angle wide. The clips were displayed by a MATLAB program using the Psychophysics Toolbox (Psychophysics Toolbox Version 3 (PTB-3) is a set of MATLAB functions for vision research). An experimenter gave the instructions, and was in the room during data collection, but the MATLAB program automatically displayed the prompts after viewing a clip. The prompts were the same as for the crowdsourced study. The spoken responses to each prompt were recorded using a headset microphone, and later transcribed, using MacSpeech Scribe v1.1 (2010) to produce the initial transcript and a separate group of Mechanical Turk workers to correct the automated transcript. Each participant viewed and responded to 40 clips randomly selected from the set of 200 clips, for a total of 2400 responses (exactly 12 per clip).

The text of responses were processed with the Text to Matrix Generator toolbox for MATLAB (Zeimpekis D, Gallopoulos E. Design of a MATLAB toolbox for term-document matrix generation. Proc. Workshop on Clustering High Dimensional Data and its Applications: SIAM; 2005:38-48.), which included a step which deleted a list of stopwords, that is, words that carry little information on their own, such as “the” and “but.” To the default stopwords list, verbal interjections were added, such as “yeah” and “um.” The toolbox converted the compiled responses to term-document matrices for numerical analysis. The matrices were used to compute the number of words in responses, and the relationship between demographics and number of words in responses. In addition, the content was evaluated by comparing responses to other responses that were made to the same video clip, or to responses to other video clips. If a response contains accurate content about the clip, then on average it should be more similar to the responses to the same video clip than it is to responses to other video clips.

The method used to compare responses was to count the number of non-repeating words that two responses had in common (after removing frequently-occurring words). More sophisticated approaches, for example that took into account synonyms, did not score as well in the validity benchmarks of example 1.

This analysis was carried out within the in-lab database, and within the Mechanical Turk database. The similarity of the two response databases was then evaluated by crossing the databases: comparing responses from one database to the responses of the other database that originated from the same video clip. The more similar the databases, the more similar responses from one database will be to responses of the other database to the same clip. Finally, the two databases were pooled and the shared words for each response, for the same clip and other clips, was computed relative to this pooled database.

Data collection for the 60 in-lab participants took place over 6 months. One subject had a cataract in one eye, one had red-green color vision deficiency, and one had early cataracts in both eyes. Data collection for the crowdsourced responses took place over 34 days of active data collection (over a 38 day period). There were 99 distinct Mechanical Turk worker IDs, which was assumed to correspond to 99 subjects. However, it is possible for a worker to create multiple accounts, with the use of additional credit cards and email addresses. The number of responses contributed by a participant ranged between 1 and 188 (median 22), usually split across multiple working sessions.

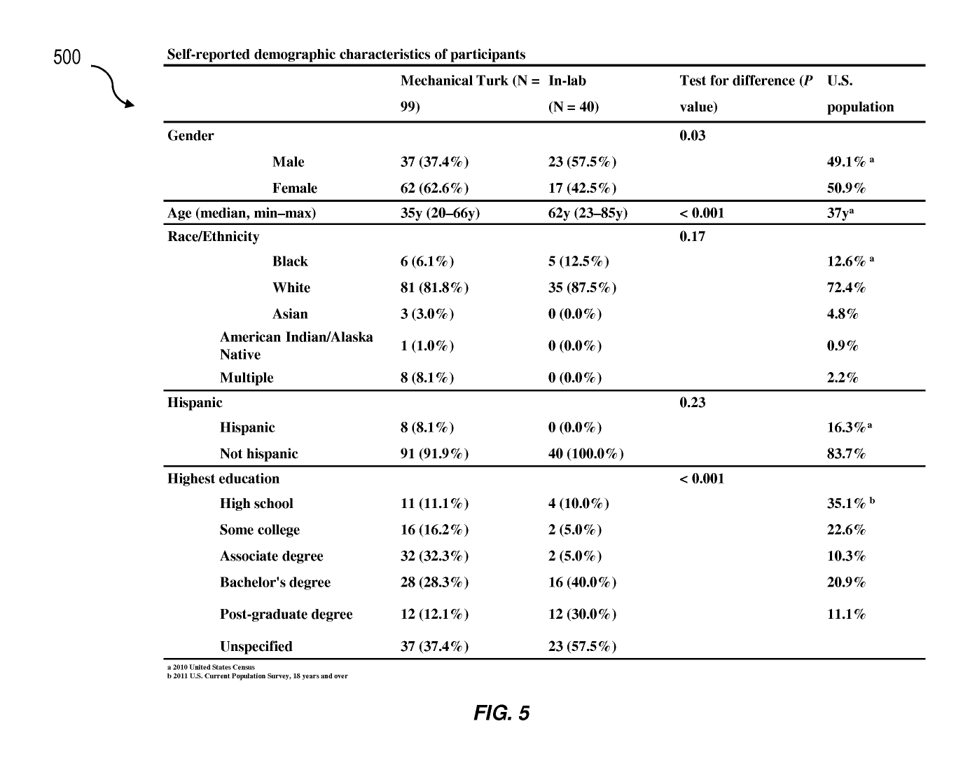

FIG. 5 is a table 500 comparing the demographics of the two control samples, and the control samples to the demographics of the United States as a whole. The crowdsourced population was skewed towards women, in a 2:1 ratio, whereas equal numbers of men and women were recruited for the in-lab study (by study design). The crowdsourced population distribution had a younger median age, but with a long tail of olderworkers (skewness=0.65). There was no significant evidence for a difference in the racial makeup of the two groups, although the proportion of people who reported their ethnicity as “Black” was twice as high in the in-lab sample compared to the crowdsourced sample (12% vs 6%), and none of the in-lab sample reported their ethnicity as “Multiple”, in contrast to 8% of the crowdsourced population. There were more people who reported themselves as “Hispanic” in the crowdsourced population. The in-lab population was more highly educated, with a greater proportion of people with Bachelor's degrees and post-graduate degrees as their maximum attainment, and a smaller proportion with a maximum attainment of “Associate degree” or “some college”.

The in-lab population was older on average than the population of the United States, whereas the median age of the crowdsourced population was 35y, only two years younger than the population of the United States (2010 census). Both populations resembled the United States in their ethnic makeup to some degree, with the greatest discrepancy from the country as a whole being in fewer Asian people, and fewer Hispanic-identified people. More people reported their ethnicities as “Multiple” in the crowdsourced population than in the population as a whole, although this may have been a result of the lack of an “Other” option. Both of the population samples had achieved a higher level of education on average than the population of the U.S. (based on people 18y and over in the 2011 Current Population Survey): They had a higher rate of Bachelor's degrees, and a lower number who had only attained high school diplomas. This could have been partly due to the greater concentration of older adults in the samples, with few participants in the 18-22y range.

The self-reported demographics of the Mechanical Turk sample are similar to those found in a survey of Mechanical Turk workers taken in 2009. Like the workers of the present example, the U.S. workers in that study had a mean age of approximately 35 years, had a large majority of women, and had approximately 40% Bachelor's degree holders, and approximately 15% with a post-graduate degree. Therefore, the sample likely represents a typical pool of U.S. participants that researchers can recruit for a study such as this through Mechanical Turk.

The two sets of participants differed somewhat in their TV and movie viewing habits and in the difficulties they experienced viewing them. There was some evidence that crowdsourced participants watched more TV, χ²(5)=11.7, p=0.04, with 38% reporting three or more hours a week compared to 19% in the in-lab sample. Crowdsourced participants also reported less difficulty with watching television (χ² (3)=11.7, p=0.04), with 84% answering “never” or “rarely” to the difficulty question compared to 72% of the in-lab participants. Far more crowdsourced participants reported having watched TV or movies on portable devices such as a cellphone than in-lab participants, 50% compared to 17%, but for those subjects who did view media on portable devices, the level of difficulty reported was not significantly different between the groups, χ² (3)=0.6, p=0.90. Crowdsourced participants watched movies in the theater somewhat more often, χ²(6)=14.2, p=0.03, with 38% watching a movie once a month or more, compared to 23% of the in-lab participants, but there was not significant evidence of a difference in the reported difficulty of watching movies, χ²(3)=7.2, p=0.06, with most crowdsourced and in-lab participants (83% and 90% respectively) reporting difficulties “never” or “rarely”.

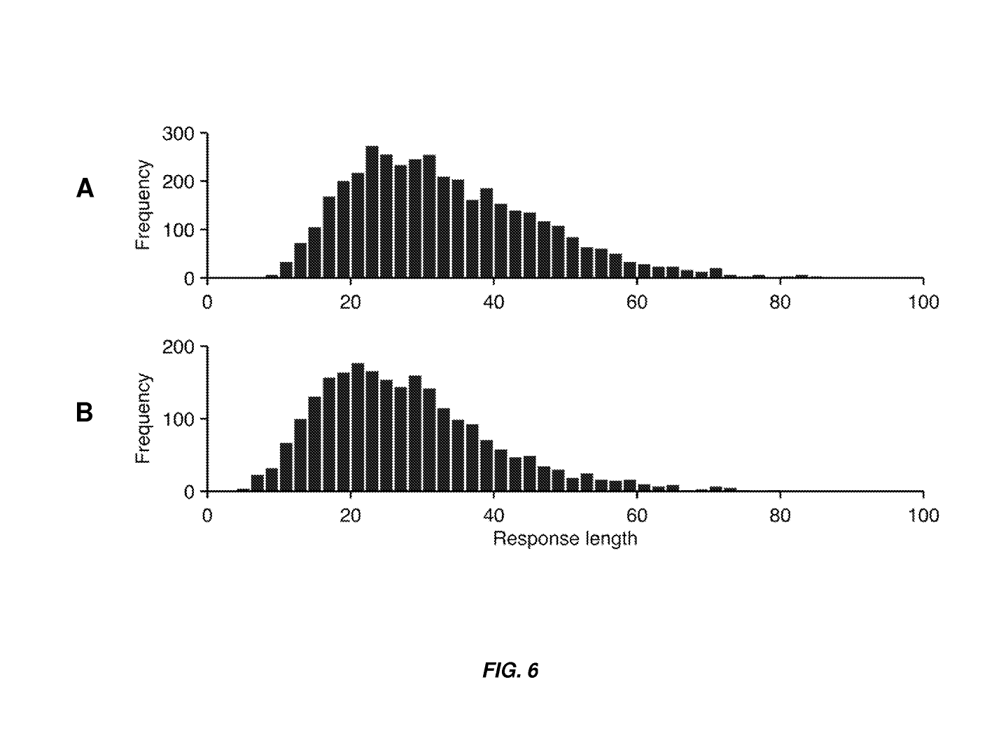

FIG. 6 are plots A and B illustrating the distribution of response lengths and their large overlap, after removing frequently-occurring words, between the in-lab and crowdsource responses. The means were significantly different, t(5318)=9.1, p<0.001, with the in-lab responses having 4 fewer words on average (M=33.2 vs M=29.1, medians 31 and 26).

The total vocabulary used in the crowdsourced responses was 8512 words for 4000 responses, whereas for the in-lab it was 5504 words for 2400 responses. They had 3965 words in common, with 4547 words appearing in the crowdsourced database but not the in-lab database, and 1539 words appearing in the in-lab database but not the crowdsourced database. The average word length was 4.1 letters for the crowdsourced data, and 4.1 letters for the in-lab data.

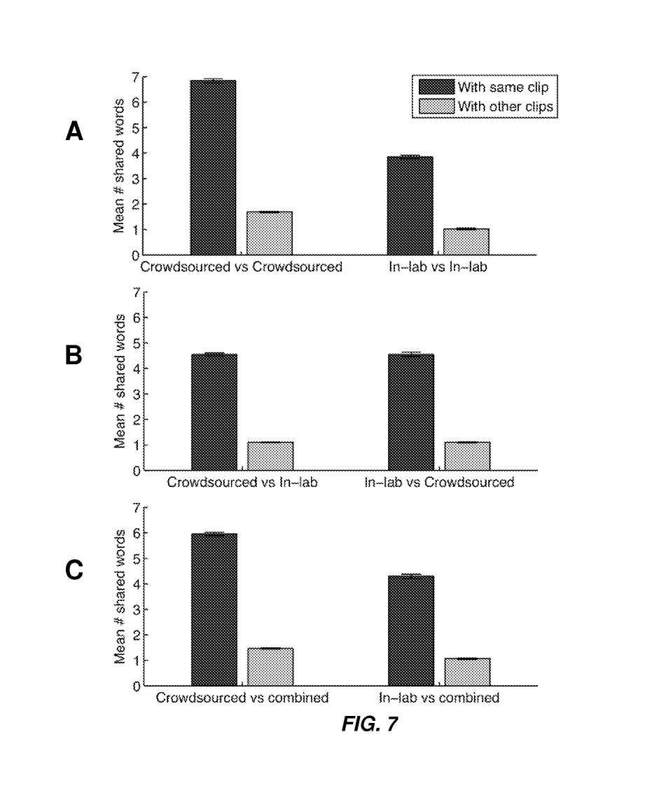

FIG. 7 shows plots A-C illustrating the mean number of words shared by responses with responses to the same clip (filled bars), and with responses to other clips (open bars). Plot A shows that comparisons occur within the same database. Plot B depicts that comparisons occur across databases. Plot C depicts that comparisons are to the combined database. Error bars indicate 95% confidence intervals.

Within each database, the words in common (after removal of stopwords) were counted with responses to the same movie clip or to other movie clips. FIG. 6A illustrates that in both databases, the similarity to responses to the same movie clip was far greater than to responses to other movie clips, with approximately twice as many shared words, F(1, 10636)=11209.8, p<0.001. There was also a difference due to the database, with the crowdsourced having larger shared word scores on average than the in-lab F(1, 10636)=2120.9, p<0.001. There was an interaction between same/other comparisons and database, F(1, 10636)=880.3, p<0.001, with the difference between the same video and other videos being larger in the in-lab condition, although the ratios were similar (4.0 in the crowdsourced condition and 3.7 in the in-lab condition).

The similarity of the two databases was evaluated by performing the same response comparisons across databases. So, a response from the in-lab database would be compared to the responses to the same movie clip in the crowdsourced database, and to responses to other movie clips in the crowdsourced database. Plot B (with reference to FIG. 6) demonstrates that the responses to the same clips were much more similar on average, t(3999)=129.6, p<0.001. Similarly, responses in the crowdsourced database were compared to responses to the same and other clips in the in-lab database, and the responses to the same clip were much more similar, t(1319)=66.2, p<0.001. Therefore the two databases were pooled and it is shown that responses were much more similar to responses to the same clip than they were to responses to other clips, F(1, 10636)=12402.3, p<0.001, and again that crowdsourced responses had higher numbers of shared words on average, F(1, 10636)=953.0, p<0.001, and also a larger difference between same-clip and other-clip shared words, F(1, 10636)=342.8, p<0.001.

Finally, it was examined whether the average shared word score for a particular clip (an indicator of the homogeneity of responses to a clip) was similar within each of the baseline databases. There was a strong correlation, r=0.75, p<0.001, between a video clip's shared word score in the crowdsourced database and in the in-lab database, indicating that clips that elicited a large amount of common vocabulary across respondents did so in both databases.

An analysis was conducted to determine whether age, gender, or maximum education level had an effect on average number of shared words or on length of responses, using mixed models with subject and video as fully-crossed random factors. In the crowdsourced responses, there was strong evidence that gender predicted shared word score, p=0.004, with men having a shared word score that was −0.61 lower on average. Age was also a significant predictor of shared word score, p=0.014, with age in years positively related to shared word score with coefficient=0.027. Education level did not significantly predict shared word score, p=0.14. None of the demographic factors significantly predicted the total number of words in responses.

In the in-lab responses, age in years predicted shared word score, p<0.001, but with a negative coefficient, −0.046. Gender and education did not significantly predict shared word score for the in-lab responses. As with the crowdsourced responses, none of the demographic factors significantly predicted the total number of words.

Example 2 shows that crowdsourced natural language responses can have substantial content, and be similar to responses obtained in the laboratory. Although the demographic characteristics were somewhat different between the two samples, with the crowdsourced population being younger, less educated, and more female, there was a large overlap in the lengths of responses that participants provided, and in the vocabulary they used to describe specific movie clips. This makes crowdsourcing a feasible approach for collecting a large control free text database, such as for use with automated natural language scoring methods.

The crowdsourced population sample resembled previous descriptions of the U.S. Mechanical Turk population, and somewhat resembled the population of the United States as a whole. The biggest distinctive feature of the crowdsourced population was the greater proportion of female participants. Based on the correlation of gender and word count, this may be the cause of the longer responses in the crowdsourced population than in the in-lab population, which had equal men and women. This could also explain the effect of gender on shared word score in the crowdsourced but not the in-lab participants: with more responses by women for comparison, in the crowdsourced sample the take-one-out procedure would score female responses higher, if there were any systematic differences between the genders.

The crowdsourced participants also watched more television and movies, and far more video on handheld devices. This reflects a greater engagement with technology, which is consistent both with a younger average age and with participation in Web-based crowdsourcing. However there was only a limited difference in the difficulty the two population samples reported in viewing video via different display devices, with both reporting the most difficulty with viewing on handheld devices, and the least difficulty with viewing movies in the theater.

Apart from the length of responses, the content was more consistent in the crowdsourced database, indicated by the larger number of shared words with responses to the same clip. This may have been due to the difference in age range between the two groups, and to the fact that responses were spoken by in-lab participants whereas the crowdsourced participants typed them. In support of the first point, which is based on the fact that ⅓ of the in-lab sample was over 70 whereas none of the crowdsourced participants were over 70, examination of the words that appeared in one sample but not the other showed likely age-related vocabulary differences, such as “fella” in the in-lab sample and “cgi” in the crowdsourced sample. This would also explain the negative relationship between age and shared word score in the in-lab database. In the crowdsourced database, a small number of outliers accounted for the appearance of a positive relationship between age and shared word score. They were subjects primarily between the ages of 20 and 30 with low shared word scores. The fact that responses were spoken could also have encouraged less formal, more idiosyncratic ways of expressing the content of the clips, which also would have reduced the mean number of shared words. Both of these possibilities predict that the shared word score with responses to clips other than the originating clip should also be lower in the in-lab sample, and this is what was observed at FIG. 6. Overall, the comparison of the two databases shows no evidence that the crowdsourced responses were of lower quality or represented less effort.

Data collection using Internet crowdsourcing took only a fraction of the time it took to recruit the target number of in-lab participants, and was less expensive when experimenter hours are considered. More studies are required to know how well these results generalize to crowdsourcing platforms other than Mechanical Turk, and to other data collection purposes. The task involved watching clips from Hollywood films, and so may have generated more engagement, and attracted more workers, than the typical survey-based Mechanical Turk study. However, good results were had in using Mechanical Turk in combination with computer speech recognition to quickly and cheaply create transcripts of the spoken in-lab responses. There is an initial cost in terms of time and technical expertise to prepare a Mechanical Turk study, and data collection is not entirely hands-off, since it is necessary to review and approve submitted work and to communicate with workers. In another natural language project using Mechanical Turk, it was detected that an individual had set up a secondary account, which became evident from the similarity of the responses, and this issue had to be resolved. However, the time investment was still much less than what is required for an in-lab study, which includes the time to identify, contact, and schedule participants in addition to their time contributing data. Altogether, these steps took more than 3 hours per participant on average, compared to only a few minutes per additional participant in the Mechanical Turk study.

A limitation of example 2 noted above is that the domain of the responses was free descriptions of short movie clips. Additional phenomena will likely be observed within natural language databases for different tasks, such as reading, smell or sound-scape description. Additionally, depending on the purpose of the data, responses may require different analysis techniques, which could increase the importance of the differences due to crowdsourcing that was found. For example, if responses are to be automatically scanned for a predefined list of keywords, then the increased probability of spelling errors when responses are typed could affect the results, as could the different vocabularies of the sets of participants. The fact that the two databases differed both in their recruitment and in their means of data collection (typed or spoken) meant that differences could not be conclusively attributed to one or the other cause. However, the results show that neither difference led to a drastic change in the lengths or vocabulary of the responses. Finally, only a simple method of scoring responses by counting the mean shared vocabulary with other responses to the same clip is reported. More sophisticated methods of scoring responses could more sensitively reveal the particularities of the databases.

Crowdsourcing can be an effective way to obtain control natural language data quickly and inexpensively, and to provide an important complement to more narrowly targeted traditional recruiting and data collection methods.

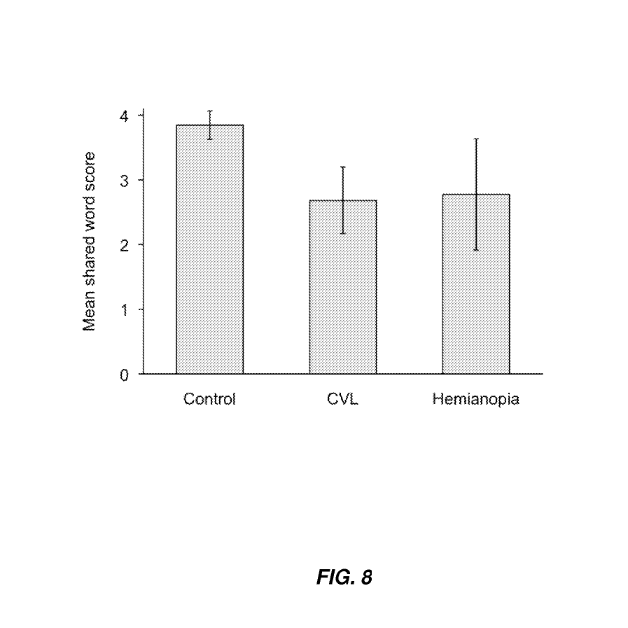

The following description provides a third example related to measuring information acquisition for patients or subjects with vision impairment. The third example compares the information acquisition of patients with central vision loss (CVL) and patients with hemianopia to the 60 control subjects who contributed responses in the laboratory for Example 2. 11 CVL patients and 7 hemianopia patients viewed 20 video clips and provided verbal responses based on the same prompts as in Examples 1 and 2, and these responses were transcribed. Both groups had significantly lower shared word scores than the control subjects, p<0.001 and p<0.05 respectively, as illustrated in FIG. 8. These results show that the current method is an effective approach to diagnosing disorders in visual and cognitive functioning.

Various implementations of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the subject matter described herein may be implemented on a computer having a display device (e.g., a CRT (cathode ray tube) or LCD (liquid crystal display) monitor) for displaying visual information and/or speakers for presenting auditory information and/or a haptic display for tactile information to the user and a keyboard and a pointing device (e.g., a mouse or a trackball) by which the user may provide input to the computer. Speakers, headphones or other sound-producing devices could be used in combination with other devices for presentation of auditory stimuli. Other kinds of devices may be used to provide for interaction with a user as well; for example, feedback provided to the user may be any form of sensory feedback (e.g., visual feedback, auditory feedback, or tactile feedback); and input from the user may be received in any form, including acoustic, speech, or tactile input.

The subject matter described herein may be implemented in a computing system that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components. The components of the system may be interconnected by any form or medium of digital data communication (e.g., a communication network). Examples of communication networks include a local area network (“LAN”), a wide area network (“WAN”), and the Internet.

The computing system may include clients and servers. A client and server are generally remote from each other and typically interact through a communication network. The relationship of client and server arises by virtue of computer programs running on the respective computers and having a client-server relationship to each other.

Although a few variations have been described in detail above, other modifications are possible. For example, the logic flow depicted in the accompanying figures and described herein do not require the particular order shown, or sequential order, to achieve desirable results. Other embodiments may be within the scope of the following claims.

1. A method comprising:
providing a stimulus;
recording a free recall response to a stimulus by a subject; and
determining, by a processor, a similarity between the recorded free recall response and one or more control responses associated with the stimulus, wherein a higher similarity indicates greater information acquisition by the subject and the stimulus is selected from a group consisting of: video, audio, image, text that is written, text that is spoken, text presented as Braille, text presented as a Rapid Serial Visual Presentation, and text presented in code.

2. The method of claim 1, wherein the stimulus is one or more of a visual stimulus, an auditory stimulus, an olfactory stimulus, and a tactile stimulus.

3. The method of claim 1, wherein the assessment of acquisition of information is used for one of the following: assessment of the subject's high-level vision; assessment of conditions that impair vision; assessment of conditions that impair hearing; assessment of conditions that impair olfaction; assessment of conditions that impair tactile sensation; assessment of conditions that impair cognitive function; assessment of treatments of vision disorders; assessment of treatments of hearing disorders; assessments of treatments of olfactory disorders; assessment of treatment of tactile disorders; assessment of treatments of cognitive disorders; assessment of a quality of the stimulus; assessment of the subject's affinity for the stimulus; and assessment of an effectiveness of compression algorithms.

4. The method of claim 1, wherein the assessment is used to evaluate interest in the stimulus.

5. The method of claim 1, wherein the similarity is determined using natural language processing.

6. The method of claim 5, wherein the similarity is determined by counting a number of words in the recorded free recall response that are contained in the database of control responses, a higher count indicating a greater similarity and a greater acquisition of information.

7. The method of claim 1, wherein the similarity is used to evaluate one or more characteristics of the subject.

8. The method of claim 7, wherein the evaluation is one or more of the following: an assessment of visual function; an assessment of a disorder affecting visual function; an assessment of auditory function; an assessment of a disorder affecting auditory function; an assessment of olfactory function; an assessment of a disorder that affects olfactory function; an assessment of tactile sensory function; an assessment of a disorder that affects tactile sensory function; an assessment of cognitive function; an assessment of a disorder affecting cognitive function; an assessment of the outcome of a medical intervention; and an assessment of attention.

9. The method of claim 8, wherein the disorder affecting visual function is selected from a group consisting of: tears, cornea, conjunctiva, crystalline lens, retinal degeneration, subretinal degeneration, dry eye, cataract, glaucoma, amblyopia, macular degeneration, retinitis pigmentosa, diabetic retinopthy, optic neuritis, acquired brain injury, and traumatic brain injury.

10. The method of claim 8, wherein the disorder affecting hearing function is selected from a group consisting of: tinnitus, sensorineural hearing loss, vestibulocochlear nerve damage, conductive hearing loss, sensorineural hearing loss, central hearing loss, functional hearing loss, and mixed hearing loss.

11. The method of claim 8, wherein the disorder affecting olfaction can be selected from a group consisting of: anosmia, dysosmia, hyperosmia, hyposmia, olfactory reference syndrome, parosmia, and phantosmia.

12. The method of claim 8, wherein the disorder affecting tactile function is selected from a group consisting of: nerve damage, allodynia, and hyperalgesia.

13. The method of claim 8, wherein the disorder affecting cognitive function is selected from a group consisting of: autism, dyslexia, dyscalculia, attention deficit disorder (ADD), schizophrenia, multiple sclerosis, stroke, mild cognitive impairment, dementias, Alzheimer's disease, acquired brain injury, and traumatic brain injury.

14. The method of claim 1, wherein the similarity is used to evaluate one or more characteristics of the stimulus.

15. The method of claim 14, wherein the evaluation is one or more of the following: an assessment of image quality; an assessment of a compression algorithm; an assessment of a stimulus presentation device quality; an assessment of the effectiveness of image; an assessment of a video enhancement algorithm; an assessment of an audio enhancement algorithm; an assessment of an enhancement algorithm settings; and an assessment of the ability of a symbol to transmit its intended message.

16. The method of claim 1, wherein the recorded free recall response is an audio recording of speech that is automatically translated to text using a speech recognition program.

17. An article of manufacture comprising:
computer executable instructions stored on non-transitory computer readable media, which, when executed by a computer, causes the computer to perform operations comprising:
receiving data characterizing a free recall response of a stimulus by a subject;
determining automatically a similarity between the recorded free recall response and a database comprising one or more control responses associated with the stimulus, wherein a higher similarity indicates greater information acquisition by the subject; and
providing the similarity.

18. The article of manufacture of claim 17, wherein providing the similarity includes at least one of displaying, storing, persisting, processing, and transmitting.

19. A system comprising:
at least one data processor;
memory storing instructions which, when executed by the at least one data processor, causes the at least one data processor to perform operations comprising:
receiving data characterizing a free recall response of a stimulus by a subject and a database comprising one or more control responses associated with the stimulus;
determining automatically a similarity between the recorded free recall response and the database, wherein a higher similarity indicates greater information acquisition by the subject; and
providing the similarity.

20. An apparatus comprising:
a stimulus source for presenting a stimulus;
a free recall recorder; and
a similarity processor configured to determine a similarity between a recorded free recall response and one or more control responses associated with the stimulus, wherein a higher similarity indicates greater information acquisition by a subject associated with the recorded free recall response and the stimulus is selected from a group consisting of: video, audio, image, text that is written, text that is spoken, text presented as Braille, text presented as a Rapid Serial Visual Presentation, and text presented in code.

21. The apparatus of claim 20, wherein the stimulus is one or more of a visual stimulus, an auditory stimulus, an olfactory stimulus, and a tactile stimulus.

22. The apparatus of claim 20, wherein the assessment of acquisition of information is used for one of the following: assessment of the subject's high-level vision; assessment of conditions that impair vision; assessment of conditions that impair hearing; assessment of conditions that impair olfaction; assessment of conditions that impair tactile sensation; assessment of conditions that impair cognitive function; assessment of treatments of vision disorders; assessment of treatments of hearing disorders; assessments of treatments of olfactory disorders; assessment of treatment of tactile disorders; assessment of treatments of cognitive disorders; assessment of a quality of the stimulus; assessment of the subject's affinity for the stimulus; and assessment of an effectiveness of compression algorithms.

23. The apparatus of claim 20, wherein the assessment is used to evaluate interest in the stimulus.

24. The apparatus of claim 20, wherein the similarity is determined using natural language processing.

25. The apparatus of claim 24, wherein the similarity is determined by counting a number of words in the recorded free recall response that are contained in the database of control responses, a higher count indicating a greater similarity and a greater acquisition of information.

26. The apparatus of claim 20, wherein the similarity is used to evaluate one or more characteristics of the subject.

27. The apparatus of claim 26, wherein the evaluation is one or more of the following: an assessment of visual function; an assessment of a disorder affecting visual function; an assessment of auditory function; an assessment of a disorder affecting auditory function; an assessment of olfactory function; an assessment of a disorder that affects olfactory function; an assessment of tactile sensory function; an assessment of a disorder that affects tactile sensory function; an assessment of cognitive function; an assessment of a disorder affecting cognitive function; an assessment of the outcome of a medical intervention; and an assessment of attention.

28. The apparatus of claim 27, wherein the disorder affecting visual function is selected from a group consisting of: tears, cornea, conjunctiva, crystalline lens, retinal degeneration, subretinal degeneration, dry eye, cataract, glaucoma, amblyopia, macular degeneration, retinitis pigmentosa, diabetic retinopthy, optic neuritis, acquired brain injury, and traumatic brain injury.

29. The apparatus of claim 27, wherein the disorder affecting hearing function is selected from a group consisting of: tinnitus, sensorineural hearing loss, vestibulocochlear nerve damage, conductive hearing loss, sensorineural hearing loss, central hearing loss, functional hearing loss, and mixed hearing loss.

30. The apparatus of claim 27, wherein the disorder affecting olfaction can be selected from a group consisting of: anosmia, dysosmia, hyperosmia, hyposmia, olfactory reference syndrome, parosmia, and phantosmia.

31. The apparatus of claim 27, wherein the disorder affecting tactile function is selected from a group consisting of: nerve damage, allodynia, and hyperalgesia.

32. The apparatus of claim 27, wherein the disorder affecting cognitive function is selected from a group consisting of: autism, dyslexia, dyscalculia, attention deficit disorder (ADD), schizophrenia, multiple sclerosis, stroke, mild cognitive impairment, dementias, Alzheimer's disease, acquired brain injury, and traumatic brain injury.

33. The apparatus of claim 20, wherein the similarity is used to evaluate one or more characteristics of the stimulus.

34. The apparatus of claim 33, wherein the evaluation is one or more of the following: an assessment of image quality; an assessment of a compression algorithm; an assessment of a stimulus presentation device quality; an assessment of the effectiveness of image; an assessment of a video enhancement algorithm; an assessment of an audio enhancement algorithm; an assessment of an enhancement algorithm settings; and an assessment of the ability of a symbol to transmit its intended message.

35. The apparatus of claim 20, wherein the recorded free recall response is an audio recording of speech that is automatically translated to text using a speech recognition program.

36. An apparatus comprising:
means for providing a stimulus;
means for recording a free recall response to a stimulus by a subject; and
means for determining a similarity between the recorded free recall response and one or more control responses associated with the stimulus, wherein a higher similarity indicates greater information acquisition by the subject and the stimulus is selected from a group consisting of: video, audio, image, text that is written, text that is spoken, text presented as Braille, text presented as a Rapid Serial Visual Presentation, and text presented in code.
